SEQUENCE LISTING (5 sequences)

SEQ ID NO: 1 - DNA, 214 bp, Infectious Hypodermal and Hematopoietic Necrosis Virus
caccatcgggcgcggatcctgcgagcgcttcgcagaaaccgttacaacctatgacgtcataggtcctatataagagatgacggactcaccggtctcccagtttctaactgacgagtgaagaggctattccaagtgctagcccgggctcgagatctgcgatctaagtaagcttggcattccggtactgttggtaaaggatcccggtccgaagcgc

SEQ ID NO: 2 - DNA, 50 bp, Infectious Hypodermal and Hematopoietic Necrosis Virus
cctgacaagacaaataaaaggcctctcagacaggatgaacagccagtacg

SEQ ID NO: 3 - DNA, 360 bp, Infectious Hypodermal and Hematopoietic Necrosis Virus
atggcaatcaatacctagtccgtcattatttggatcatcaagtaacagtgaaccatcagaagtctttcaaaacgtcttcggggaaagacaaaccgaaggatacaaatgtaagtacaagtgactgactaagtgacgatccattaaattcctaattgacgcaagtgacgacgtcatatgcgtcacttacaaaagacgtaaccgcattcgtccatcactcacatatatctttctctacctttcagacgacataccccaacaaatatcgctgcgctactgcccagatcacattctaccgtggtgcttcatagggaacagacccgttctctactgcctctgcaacgagtgttttatagacaatct

SEQ ID NO: 4 - DNA, 35 bp, Artificial Sequence
  Synthetic construct
cgcggatccgaacactgcgtttgctggctttgatg

SEQ ID NO: 5 - DNA, 27 bp, Artificial Sequence
  Synthetic construct
gtccaccaatagatgtcgagtctacgg

(19) United States
(12) Patent Application Publication
(10) Pub. No.: US 2012/0202249 A1
(43) Pub. Date: Aug. 9, 2012

(54) PRODUCTION OF AN INTACT VIRUS IN A MAMMALIAN (NON-HOST) CELL SYSTEM USING A SECONDARY NON-HOST VIRAL CONSTRUCT

(76) Inventor: Arun K. Dhar, Sykesville, MD (US)

(21) Appl. No.: 13/263,360

(22) PCT Filed: Apr. 8, 2010
(86) PCT No.: PCT/US10/30348

§ 371 Date: Mar. 19, 2012

Related U.S. Application Data
(60) Provisional application No. 61/167,846, filed on Apr. 8, 2009.

(51) Int. Cl. C12P 21/00; C12N 7/00; C12N 5/10; C12N 15/86; C12N 15/866

(52) U.S. Cl. 435/69.3; 435/320.1; 435/348; 435/358; 435/325; 435/365; 435/364; 435/235.1

(57) ABSTRACT

The present invention relates to constructs and methods for the production of recombinant proteins, viruses, and viral vaccines in heterologous culture systems by expressing intact genes or viral genomes under the control of a pantropic promoter in a culture system that is not considered a host to the virus so produced. The promoter/viral genome constructs are inserted into a baculovirus and expressed in mammalian non-host cells for the baculovirus.

### CROSS-REFERENCE TO RELATED APPLICATION

This application claims priority to U.S. Provisional Application No. 61/167,846 filed in the United States Patent and Trademark Office on Apr. 8, 2009, the contents of which are hereby incorporated by reference herein for all purposes

### BACKGROUND OF THE INVENTION

1. Field of the Invention

The present invention relates generally to an expression system, and more specifically, a method and system for rapidly producing large quantities of a viral vaccine, such as an attenuated Hepatitis C viral vaccine, using a nonhuman cell production system and a virus carrying a pantropic promoter driving the expression of the attenuated Hepatitis C viral vaccine in that non-human production system.

2. Related Background Art

Shrimp represents an aquaculture crop of increasing importance, particularly to countries with coastal environments within 30 degrees North and South latitude from the equator. Accompanying the expansion and intensification of this industry, there has been a growing knowledge base on viral and bacterial diseases that can affect shrimp and potentially limit productivity. Since the first report of a viral disease in early 1970s, more than 20 viruses have been reported that infect shrimp (Lightner, 1996) and this list is growing rapidly. The four most important viruses of penaeid shrimp are white spot syndrome virus (WSSV), yellowhead virus (YHV), Taura syndrome virus (TSV), and the infectious hypodermal and hematopoietic necrosis virus (IHHNV). All four viruses have now been sequenced (Shike et al., 2000, van Hulten et al., 2000, Yang et al., 2001, Mari et al., 2002, Sittidilokratna et al., 2008).

Dhar and Allnutt, recently described how a promoter, identified from one of these shrimp viruses (IHHNV), could be used in conjunction with an Internal Ribosomal Entry Site (IRES) element to create an expression vector for the transient expression of foreign genes, and for the transfection of shrimp primary cell lines with foreign genes or modifiers of endogenous genes (Dhar and Allnutt, 2004). These vectors could be used (1) to express recombinant protein(s) with therapeutic potential using shrimp, (2) to express host gene or foreign gene in excess to determine their role in growth, development, and or disease resistance using shrimp, (3) to develop a transgenic shrimp, and (4) to study the role of virally encoded protein in viral pathogenesis in vitro and in vivo in shrimp.

The expression of genes in heterologous systems is generally more problematic than in homologous systems, particularly if the gene source and the heterologous system are far apart phylogenetically. Issues of codon usage, specific and unique regulatory sequences, and post-translational modifications all need to be considered when using heterologous systems production systems.

The production of attenuated viruses or recombinant vaccines for the Hepatitis C virus (HCV) has long been problematic. HCV is a small (50 nm in size), enveloped, single-stranded, positive sense RNA virus and is the only known member of the Hepacivirus genus in the family Flaviridae. Its presence was known as early as the 1970s as a post-transfusion hepatitis that was identified as non-A non-B hepatitis, but it was only confirmed that this was HCV by Kuo and colleagues in 1989 (Kuo et al., 1989). There are now known to be six major genotypes of HCV and at this time there is still no vaccine for any of these genotypes. It is estimated that 150-200 million people worldwide are infected with HCV (for review see http:///en.wikipedia.org/wiki/Hepatitis C).

Thus, it would be advantageous to develop a system to express genes in a heterologous system to develop a vaccine that could have an impact on the world wide infection of HCV.

### SUMMARY OF THE INVENTION

The present invention is based on the discovery of the pantropic nature of a known shrimp viral promoter and that this promoter could be used to express an entire viral genome in a heterologous system. This discovery leads to the development of a novel approach for the production of a mammalian virus in an insect or mammalian cell culture using a heterologous expression vector, by demonstrating the use of the pantropic shrimp viral promoter discovered by the inventors to drive the expression of a shrimp single stranded RNA viral genome in a baculovirus backbone in insect and mammalian cell lines.

The present invention has particular relevance to the development of new HCV vaccines (attenuated live virus, defective interfering particles (DIPs), or recombinant vaccines expressed in mammalian cells). The process and construct can also be used for the production of a number of other vaccines and recombinant proteins for diagnostic or therapeutic use in animals including man.

The instant invention is based on the discovery of the pantropic nature of a known shrimp viral promoter and its use to express an entire viral genome in a heterologous system. This discovery leads to the development of a novel approach for the production of a shrimp virus in an insect cell culture using a heterologous expression system. Specifically, the invention involves the use of the viral promoter of Infectious Hypodermal and Hematopoetic Virus (IHHNV) to drive the expression of a shrimp single stranded RNA viral genome using a baculovirus vector in insect cell lines.

In one aspect the present invention relates to the discovery that the pantropic nature of a viral promoter of (IHHNV) can be used to drive the expression of an entire first viral genome or parts thereof when inserted (inclusion virus) into a second viral genome (carrier virus construct), thereby resulting in the simultaneous production of two viruses in one cell type.

In another aspect, the present invention relates to the expression of a viral genome that is non-infective to the cell type wherein the recombinant virus is being made.

In yet another aspect, the present invention relates to the expression of a gene coding for proteins that are heterologous to the production cell type itself by using the pantropic promoter described herein.

In one aspect, the present invention relates to a viral construct comprising a carrier virus and at least one viral promoter of IHHNV operably linked to at least one inclusion viral genome, gene or fragments thereof, wherein the inclusion viral genome is different from that of the carrier virus. Preferably the viral promoter of IHHNV is selected from the group consisting of P2 or P61. Preferably the viral promoter of IHHNV is selected from the group consisting of P2 (SEQ ID NO.: 1) and P61 (SEQ ID NO.: 2) of AF273215. Other promoter regions for P2 of IHHNV include: 61-110 of AF27315; 217-266 of Ay095351; 289-338 of X74945; 261-310 of M37899; 152-203 of AY578734; and 133-181 of NC 007218. Other promoter regions for P61 of IHHNV include: 2398-2447 of AF273215; 2399-2448 of AY095351; 2499-2548 of NC 004285; 2363-2412 of M37899; 108-152 of DQ206403; and 3359-3431 of NC 007218.

In yet another aspect, the present invention provides for a method to produce a functional virus in a heterologous system, the method comprising:

preparing a full length DNA sequence of a virus;

linking an IHHNV promoter sequence upstream of the 5′ end of the DNA sequence to drive the transcription of the DNA sequence;

inserting the linked sequences into a transfecting virus; and

infecting a host cell for expression therein of at least the linked sequences.

The method can include both RNA and DNA viruses and the preferably the IHHNV promoter is selected from the group consisting of P2 or P61.

In yet another aspect, the present inventions provides for a method of expressing recombinant proteins in a heterologous culture system, the method comprising:
preparing a nucleotide sequence encoding the proteins,
linking an IHHNV promoter sequence upstream of the 5′ end of the nucleotide sequence encoding the proteins to drive the transcription of the nucleotide sequence and forming a linked sequence;
inserting the linked sequence into a transfecting carrier virus; and
infecting and culturing a host cell for expression therein of the transfecting carrier virus and proteins.

In a further aspect, the present invention provides for a host virus that has the ability to express its own genomic material and that of a second virus, wherein the second virus genes or viral genomes are under the control of a pantropic promoter of IHHNV in a culture system that is not considered a host to the second virus.

These and other aspects of the present invention will be apparent from the detailed description of the invention provided hereinafter.

### BRIEF DESCRIPTIONS OF THE FIGURES

FIG. 1 shows the acute phase TSV infection in P. vannamei shrimp (a representative sample from Bioassay #3). The panel on the left shows H&E of P. vannamei stomach epithelium, and the panel in the right shows in situ hybridization of P. vannamei stomach epithelium. The positive signal in in situ hybridization is indicated by black blue precipitations. Cells surrounding the black blue precipitation are healthy and did not react with TSV-specific probe.

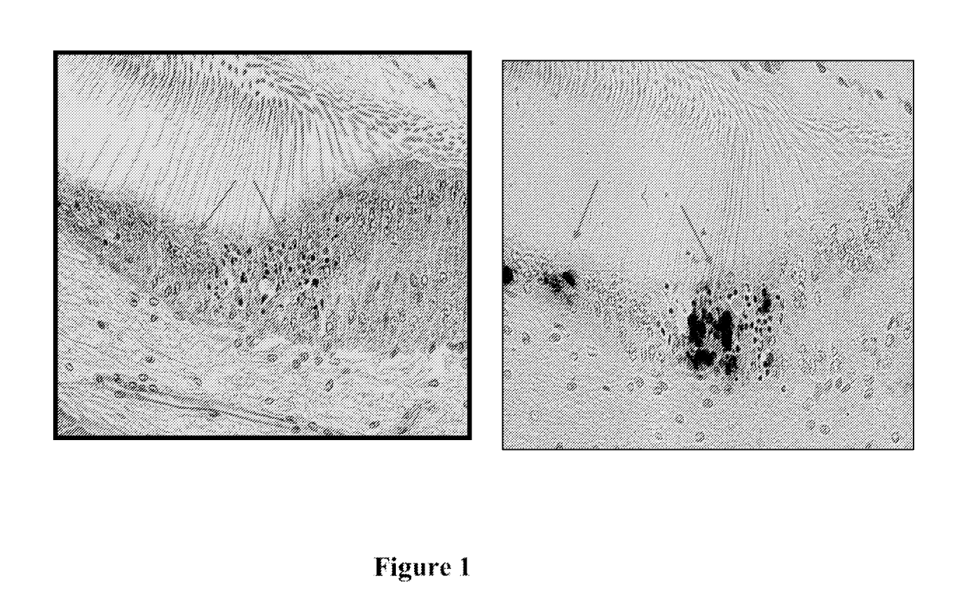

FIG. 2 shows (A) An agarose gel photograph of 5′-RACE using cDNAs from purified recombinant TSV generated in CHO cells. The arrow indicates the cDNA amplicons. (B). A schematic diagram indicating the origin of the transcripts in recombinant TSV as determined by 5′-RACE is shown in panel B. The alphabets indicate the coordinates of each transcript compared to the nucleotide position 1 in the TSV genome. A list of 5′ RACE cDNA clones sequenced and the details of the coordinates of each transcript of recombinant TSV is given in Table 1.

FIG. 3 shows the clinical sign of TSV in shrimp injected with rTSV generated in CHO cells via baculovirus mediated infection. Melanized lesions on the exoskeleton, a hallmark of TSV chronic phase infection, are clearly visible on the virus-injected (I) shrimp but not on the healthy (H) shrimp.

### DETAILED DESCRIPTION OF THE INVENTION

The inventor has discovered the pantropic nature of a shrimp viral promoter and has used it to drive the expression of a viral genome when inserted into a second viral genome, thereby resulting in the simultaneous production of two viruses in one cell type. Furthermore, the inventor discovered that the viral genome expressed using this system could be non-infective to the cell type where the recombinant virus is being made. The inventor further discovered that the pantropic promoter could also drive the expression of a gene coding for proteins that were heterologous to the production cell type itself. Specifically, the invention relates to a method for rapidly producing large quantities of a viral vaccine, such as an attenuated Hepatitis C viral vaccine, using a nonhuman cell production system and a virus carrying the pantropic promoter driving the expression of the attenuated Hepatitis C viral vaccine in that nonhuman production system.

In describing the present invention, the following terminology is used in accordance with the definitions set out below.

Following long-standing patent law convention, the terms “a” and “an” mean “one or more” when used in this application, including the claims.

The term “expression,” as used herein, refers to the transcription and translation of a structural gene so that a protein is synthesized.

The term “linked,” as used herein, refers to functional linkage between a promoter and a second sequence, wherein the promoter sequence initiates transcription of nucleotide sequences of the second sequence.

The term “polypeptide,” as used herein, refers to peptides, polypeptides and proteins, unless otherwise noted. As used herein, the terms “protein,” “polypeptide” and “peptide” are used interchangeably herein when referring to a gene product.

The term “polynucleotide,” as used herein, means a sequence of nucleotides connected by phosphodiester linkages. A polynucleotide of the present invention can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide of the present invention can be prepared using standard techniques well known to one of skill in the art.

The term “promoter” includes all sequences capable of driving transcription of a coding sequence in a cell, e.g., a plant cell, animal cell, bacterial cell, fungal cell, and yeast cell. Thus, promoters used in the constructs of the invention include cis-acting transcriptional control elements and regulatory sequences that are involved in regulating or modulating the timing and/or rate of transcription of a gene or genes. For example, a promoter can be a cis-acting transcriptional control element, including an enhancer, a promoter, a transcription terminator, an origin of replication, a chromosomal integration sequence, 5′ and 3′ untranslated regions, or an intron sequence, which are involved in transcriptional regulation. A promoter sequence can be “operably linked to” a coding sequence when RNA polymerase which initiates transcription at the promoter will transcribe the coding sequence into RNA, as discussed further, below.

The term “viral genome” includes all the viral nucleic acid that is required to produce a fully functional infective virus. The nucleic acid of the viral genome can be single or double stranded DNA or RNA.

The term “gene,” as used herein, refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression,” as used herein, refers to the process of converting genetic information encoded in a gene into RNA (e.g., mRNA, rRNA, tRNA, or snRNA) through “transcription” of a deoxyribonucleic gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (ie., RNA or protein), while “down-regulation” or “repression” refers to regulation that decrease production. Molecules (e.g., transcription factors) that are involved in up-regulation or down-regulation are often called “activators” and “repressors,” respectively.

The term “substantial identity,” as used herein means that a polynucleotide or polypeptide comprises a sequence that has at least 80% sequence identity, preferably at least 90% or more preferably at least 97%, compared to a reference sequence over a comparison window.

In addition to a promoter sequence, the expression cassette may include a transcription termination region downstream of the structural gene to provide for efficient termination. The termination region may be obtained from the same gene as the promoter sequence or may be obtained from different genes.

The vector may also typically contain a selectable marker gene by which transformed cells can be identified in culture. Usually, the marker gene will encode antibiotic resistance. These markers include resistance to G418, hygromycin, bleomycin, kanamycin, and gentamicin. After transforming the cells, those cells having the vector will be identified by their ability to grow in a medium containing the particular antibiotic.

The vectors described above can be microinjected directly into cells by use of micropipettes to mechanically transfer the recombinant DNA. The genetic material may also be transferred into the cell using polyethylene glycol. Another method of introduction of polynucleotide sequences is particle acceleration of small particles with the nucleic acid either within the matrix of small beads or particles, or on the surface. Yet another method of introduction is fusion of protoplasts with other entities, such as, minicells, cells, lysosomes or other fusible lipid-surfaced bodies. The DNA may also be introduced into the cells by electroporation wherein electrical impulses of high field strength reversibly permeabilize biomembranes allowing the introduction of the plasmids.

All other terms are defined in the literature using Sambrook et al. (1989) as a guide.

It is an object of the invention to provide a genetic construct for the expression of a foreign virus, in a cell, tissue or whole organism-based production system that is not normally infected by that virus.

It is a further object of the invention to provide a genetic construct for the expression of a foreign gene or genes, including but not limited to entire viral genomes, in a cell, tissue or whole organism-based production system that is heterologous to the gene or genes and is not normally infected by the resulting virus.

It is a further object of the invention to provide a method by which large quantities of an attenuated viral vaccine are produced for an animal including by transfection of a cell culture system with a virus carrying the attenuated viral genome whose expression is driven by a pantropic promoter.

It is a further object of the invention to provide a method by which attenuated viral vaccine constructs can be rapidly produced for screening purposes by transfection of a cell culture system with a virus carrying a number of different attenuated viral genome constructs whose expression is driven by a pantropic promoter.

Therefore, the invention provides a composition for expressing foreign gene(s) or entire viral genomes in a heterologous or typically non-infective cell culture system and a method of producing large quantities of such products that include, but are not limited by vaccines, diagnostics, and therapeutic products.

Pantropic promoter from Infectious Hypodermal and hematopoetic Virus (IHHNV) of shrimp.

IHHNV is a major viral pathogen of penaeid shrimp. IHHNV virions are icosahedral, nonenveloped, and contain a single-stranded DNA genome of 4.1 kb size. It was first detected in 1981 by Lightner and colleagues (Lightner et al., 1983). The IHHNV genome contains three large open reading frames and the genome organization has many similarities to mosquito Brevidensoviruses (Shike et al., 2000). Further analysis of this gene sequence led to the identification by the inventor of two putative promoter elements, designated P2 and P61, detected upstream of left Open reading Frame (ORF) and right ORF of the IHHNV genome (Shike et al., 2000). Both of these promoters were shown to drive the expression of firefly luciferase reporter constructs in insect cells, fish cells and in shrimp tail muscle and are therefore referred to as pantropic promoters (Dhar et al., 2007). Transcription mapping of IHHNV encoded genes revealed that the IHHNV middle ORF is transcribed by a novel promoter, named Mid, and the Mid promoter is functional in insect cells (SEQ ID NO.: 3).

Pantropic promoters such as, but not limited to, the P2 or P61 promoters of IHHNV, can be used in the practice of the instant invention. Constructs containing such promoters can be prepared by functionally linking the identified promoter region to a gene or genes, or an entire viral genome such that the expression will result in the production of functional polypeptides or virus particles. Such constructs can then be incorporated into a second vectors system, such as, but not limited to, baculoviruses, lentiviruses, adenoviruses, or plant viruses, resulting in the production of both the recombinant vector viruses as well as the constructed virus in the host organism of the second vector system. In such a way, a modified shrimp virus such as TSV or an attenuated mammalian viral vaccine (e.g., for HCV) could be manufactured in large quantity in a relatively short period of time in a baculovirus-based insect cell or insect larval production system, both of which are well known in the art.

### Taura Syndrome Virus (TSV) of Shrimp.

The TSV genome is a single-stranded RNA of positive polarity with a 3′-poly(A) tail (Bonami et al. 1997). The genome is 10,205 nucleotides (not) long with a 5′ untranslated region of 377 nt and a 3′ untranslated region of 226 nt (Mari et al. 2002). There are two open reading frames (ORFs) in the TSV genome. ORF1 is 6324 nt long, and encodes a 2107 amino acid (aa) polyprotein with a molecular mass of 234 kDa. ORF2 is 3036 nt long and encodes a 1011 aa polypeptide with a molecular mass of 112 kDa (Mari et al. 2002, Robles-Sikisaka et al. 2001). There is an intergenic region of 226 nt between the two ORFs. ORF1 encodes non-structural proteins (helicase, a protease and a RNA-dependent RNA polymerase, RdRp), and ORF2 encodes the virion structural proteins (Mari et al. 2002, Robles-Sikisaka et al. 2001). TSV virions contain three major polypeptides, designated as VP1 to VP3 (55, 40, and 24 kDa), and one minor polypeptide (58 kDa), designated as VP0 (Bonami et al. 1997). The N-termini of VP1 to VP3 have been sequenced, and the order of these proteins in ORF2 was found to be VP2, VP1 and VP3 (Mari et al., 2002). Based on the biophysical and genomic properties, TSV has been classified as a member of a newly created family, Dicistroviridae with CrPV as the type species (Christian et al., 2006).

In order to produce functional dicistrovirus in a heterologous system (e.g., TSV in insect cells) a full length DNA clone of the virus is prepared and a pantropic promoter such as, but not limited to, the P2 or P61 promoters of IHHNV is cloned upstream of the 5′ end of the sequence in a fashion that will drive the transcription of the viral genome, and the sequence is inserted into a non-coding region of a transfecting virus such as, but not limited to, baculovirus. Likewise, in order to produce functional attenuated picornaviral or coronaviral vaccines, a full length DNA clone of the attenuated virus is prepared and a pantropic promoter such as, but not limited to, the P2 or P61 promoters of IHHNV is attached to the 5′ end of the sequence, and the sequence is inserted into a non-coding region of a transfecting virus such as, but not limited to, baculovirus.

Baculovirus Expression System with Insect Cells or Larval Culture.

Baculoviruses represent a family of large, rod-shaped enveloped viruses with a double stranded DNA genome size of from 80-180 Kb. Baculoviruses are considered to be very species specific among invertebrates with over 600 host species described, but they are not known to infect mammalian or other vertebrate animal cells (for a review of Baculoviruses see: http://en.wikipedia.org/wiki/Baculoviridae). In the 1940's they were used widely as biopesticides and since the 1990's they have been used for producing complex eukaryotic proteins in insect culture cells (e.g., sf9) or insect larvae (e.g., lepidopteran larvae). The most widely studied baculovirus is Autographa californica multicapsid nucleopolyhedrovirus (AcMNPV), a 134 Kb genome virus with 154 open reading frames.

Commercially available viral expression systems, such as the baculovirus systems that are well know in the art, can be employed in the practice of the instant invention. The gene(s) or natural or attenuated viral genome/pantropic promoter constructs are inserted into the baculovirus genome in such a way that following infection of the host with the baculovirus, the heterologous viral genome is transcribed in parallel to the baculovirus genome. Upon transcription, both baculovirus RNAs as well as the heterologous viral RNA, will be translated to produce corresponding viral encoded proteins. Subsequently, mature virions of baculovirus and the RNA virus will be produced. Therefore, the product of such a system will, in fact, be two intact virons (the RNA virus and the baculovirus) but only the baculovirus and not the RNA virus will be able to infect new insect cells because insect cells are not the natural host of the RNA virus. In this way, however, the quantity of the active RNA virus is also amplified by the baculovirus cycle. If the RNA virus is an attenuated viral vaccine for use in a vertebrate, including man, the two viruses need not be isolated as the baculovirus will not infect vertebrate cells and only the attenuated viral vaccine will be effective.

### Chinese Hamster Ovary (CHO) Cell Expression System.

CHO cell cultures were established in the 1960s and are used extensively in research and for commercial production of diagnostic and therapeutic products. In fact, CHO cells are the most commonly used mammalian hosts for industrial production of recombinant protein therapeutics and viral vaccines. Although simple recombinant proteins can be produced using bacterial cultures, more complex proteins or mammalian viruses that must be glycosylated to ensure their most functional state, must be made in mammalian cells. Vaccines for polio, measles, mumps, rubella and chickenpox, for example, are currently made in mammalian cell cultures.

Baculoviruses are known to infect insects. In recent years, baculoviruses have been widely used for in vitro and in vivo gene delivery in mammalian system (Tani et al., 2003). Unlike other mammalian viruses, baculoviruses do not replicate in mammalian cells, have no marked cytotoxicity and are thus safer than conventional viral vector. As a result, baculoviruses have been successfully used in gene delivery in primary and immortal cell lines as well as for in vivo gene delivery (Tani et al., 2003). Therefore, once the baculovirus is generated in insect cells, it can be used to infect mammalian cell culture such as, but not limited to, Chinese Hamster Ovary (CHO) cells, African Green Monkey kidney (AGMK) cells, CaCo-2 cells, or COS-7 cells to deliver the foreign gene in mammalian host. When the baculovirus is modified to contain the genome of an attenuated virus of mammalian origin and used to infect mammalian cells in a cell culture system, it will result in the production of the purified attenuated viral vaccine since the baculovirus will not multiply in the mammalian cells, whereas the newly produced viral vaccine will propagate, albeit poorly if attenuated, in the mammalian cell culture.

### Hepatitis C (HCV) and Other Mammalian Viruses.

To date there is no Hepatitis C vaccine. Establishing an attenuated HCV vaccine would be of great benefit to the human population as it would be simple and easy to use (like polio vaccine). However, being attenuated generally means that it will not effectively replicate in mammalian cells making large-scale production of such a vaccine difficult. To overcome this limitation, the HCV genome, genes or fragments thereof are cloned into baculovirus under the expression control of a pantropic promoter such as, but not limited to the P2 promoter of IHHNV. Applicable HCV genomes include, but are not limited to, Accession Nos. NC 00412; NC 00927; NC 009826; NC 009825; NC 009824; NC 009823 and NC 001655. The resulting recombinant baculovirus is then used to infect insect cells or whole lepidopteran larvae with a high degree of efficiency and will generate large quantities of the attenuated HCV vaccine along with the recombinant baculovirus. The two viruses are then separated by standard processes known by those familiar with the art and can then be used for and oral or injection vaccination.

### EXAMPLES

The invention, as contemplated herein, is a composition comprising nucleotide sequence of an intact viral genome (the product virus) and a pantropic promoter, which is inserted into the genome of a second virus (the facilitating virus), which has a different host range than the product virus. The invention also provides methods for the production of a product virus, attenuated or otherwise, in a system that is not a host to the product virus and may or may not be a host to the facilitating virus. The invention also describes a method for the protection of animals, including humans, from disease by supplying a vaccine, which is a product of the invented method.

The following examples outline the invention and are used for exemplification purposes only and are not intended to limit the scope of the invention in any way.

### Example 1

### Isolation and Purification of Taura Syndrome Virus from Transfected Mammalian Cells

Recombinant baculovirus stock was used to infect mammalian cells (293H and CHO) for generating an RNA virus, TSV. Monolayers of 293H and CHO cells were infected with recombinant baculovirus containing a full-length TSV genome. Cells were infected at an MOI of 10 and incubated 72 hours to generate viral particles. Cell supernatants were removed and cells were lysed by freeze-thaw in PBS. Virus particles were isolated from supernatants as well as from cell lysates using a sucrose density gradient centrifugation. One band was observed in the density gradient that contained virions of icosahedral morphology as seen by transmission electron microscopy. These virions are similar in morphology to the wild tvae TSV.

### Example 2

### Production, Isolation and Purification of Norovirus from Transfected Mammalian Cells

In order to produce a recombinant Norovirus in mammalian cells, pantropic promoter such as IHHNV P2 promoter is cloned downstream of a commercially available baculovirus viral expression vector, such as pFastBacl (Invitrogen Inc., Carlsbad, Calif.). Subsequently the full-length cDNA of a Norovirus is cloned downstream of P2 promoter. The plasmid DNA of the bacmid clone is then be used to transfect Sf9 cells to generate a recombinant baculovirus containing a Norovirus genome. The recombinant baculovirus upon infecting mammalian cells produces Norovirus. Since mammalian cells do not support the replication of baculovirus, only Norovirus is produced.

### Example 3

### Production, Isolation and Purification of Hepatitis C Virus from Transfected Mammalian Cells

In order to produce a recombinant Hepatitis C virus (HCV) in mammalian cells, pantropic promoter such as IHHNV P2 promoter is cloned downstream of a commercially available baculovirus viral expression vector, such as pFastBacl (Invitrogen Inc., Carlsbad, Calif.). Subsequently the full-length cDNA of a HCV isolate is cloned downstream of P2 promoter. The plasmid DNA of the bacmid clone is then used to transfect Sf9 cells to generate a recombinant baculovirus containing a HCV genome. The recombinant baculovirus upon infecting mammalian cells produces mature virions of HCV. Since mammalian cells do not support the replication of baculovirus, only HCV is produced.

### Example 4

### Enhancing the production of Norovirus and Hepatitis C Virus in Mammalian Cells

The production of Norovirus and Hepatitis C virus in mammalian cell culture system is enhanced by co-infecting the cells with baculovirus containing a full-length genome or a Norovirus or HCV as well a baculovirus containing the Norovirus or HCV structural and nonstructural genes driven by two different promoters. Supplementation of the baculovirus (containing a full-length Norovirus or HCV) infection with the structural and non-structural proteins in trans (from a second baculovirus containing structural and non-structural genes) enhances the production of the heterologous virus in mammalian cell culture system.

### Example 5

### Determining the infectivity of the recombinant RNA Virus from Example 1

In order to determine the infectivity of the recombinant TSV (rTSV) generated via baculovirus mediated infection in CHO and 293H cells, the purified rTSV was injected into shrimp (P. vannamei, Kona line, Oceanic Institute, HI). Shrimp injected with 2% saline solution served as a control treatment. After injection, animals from each treatment were maintained in 20 L aquaria. Each aquarium contained artificial seawater (25 ppt) at 27-29° C., was supplied with continuous aeration through placement of a single airstone, and kept covered to prevent both shrimp escape and possible cross-contamination by aerosols. The shrimp were acclimated for 1-2 days prior to the onset of each bioassay. Animals were fed a pelleted ration (SI35 pellet, Ziegler Brothers) ad libitum twice per day during the entire duration of the study. A 50-70% water exchange was conducted on each tank, following siphoning of organic debris, every 3 days during the course of each bioassay. The shrimp were observed two to four times daily to remove and preserve moribund and dead shrimp. Mortalities in the rTSV injected group ranged from 87-94% at the termination of the experiment (7 to 10 days post-injection). There was no mortality in the control treatment. Virus injected shrimp showed clinical signs of TSV infection such as lethargy, loss of appetite, opaque musculature, and in some cases partial molting. Moribund animals also showed reddening of the roods/antennae and darkening of the body. Surviving animals displayed variable sized multifocal melanized lesions throughout the body, typical of TSV transition phase infection. None of the animals in the control group displayed any clinical signs similar to those injected with purified rTSV. This confirmed the infectivity of rTSV generated in CHO cells via baculovirus mediated transfection.

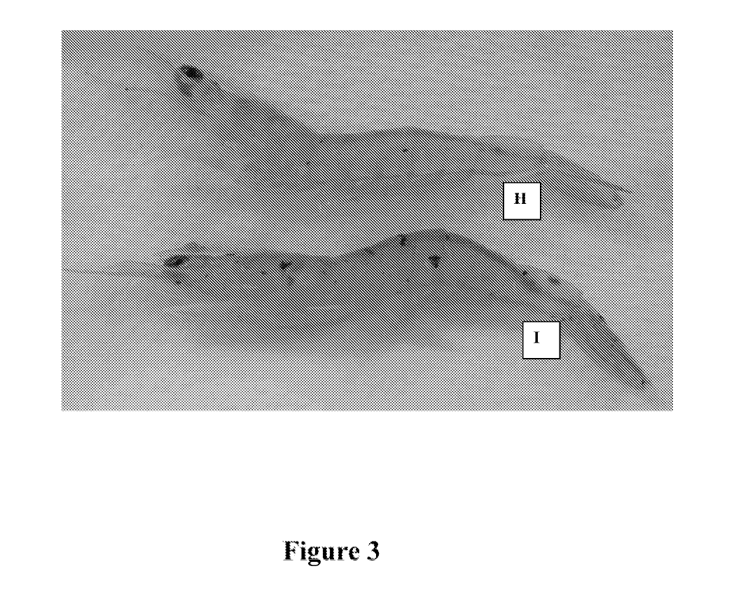

Clinical sign of TSV in shrimp injected with rTSV generated in CHO cells via baculovirus mediated infection is shown in FIG. 3. Melanized lesions on the exoskeleton, a hallmark of TSV chronic phase infection, are clearly visible on the virus-injected shrimp

Cephalothoraxes from moribund and surviving animals of rTSV injected group as well as control treatment group was preserved in Davidson's solutions for histopathology using hematoxylin and eosin (H&E) staining and in situ hybridizations using TSV-specific probe following standard protocol (Bell and Lightner, 1988). The tail muscle samples were preserved at −80° C. for isolating total RNA and mapping the 5′-end of the rTSV RNA.

Routine histological analysis of representative negative control shrimp collected upon termination of each bioassay demonstrated that they were free of TSV as well as any other known shrimp viruses. In contrast, pathodiagnostic acute, transition or chronic phase TSV lesions were detected histologically in rTSV injected shrimp. Briefly, acutely infected shrimp were characterized by the presence of multifocal epithelial necrosis within the stomach, appendages, general body cuticle, and gills; transition phase animals were those demonstrating both acute phase lesions (epithelial necrosis) and lymphoid organ spheroids with or without cuticular melanization, and chronic phase shrimp were those only demonstrating numerous lymphoid organ spheroids. In situ hybridization using TSV-specific probe confirmed TSV infection.

In order to map the 5′-end of the rTSV and to differentiate it from the wild type TSV, 5′ Rapid Amplification of cDNA End (5′ RACE, Clontech, Inc.) was performed using total RNA isolated from purified rTSV. The primers used for the 5′ RACE include a forward primer from the 5′ RACE kit (5′RACE Inner primer: 5′ CGCGGATCCGAACACTGCGTTTGCTGGCTTTGATG 3′) (SEQ ID NO.: 4) and a TSV-specific reverse primer (TSV222R: 5′ GTCCACCAATAGATGTCGAGTCTACGG 3′) (SEQ ID NO.: 5). Two amplicons, approximately 220 by and 320 by in size were obtained by RT-PCR. The amplified cDNAs were gel-purified and cloned into a pCR2.1-TOPo vector (Invitrogen, Inc.). Plasmid DNA was isolated from recombinant clones and sequenced.

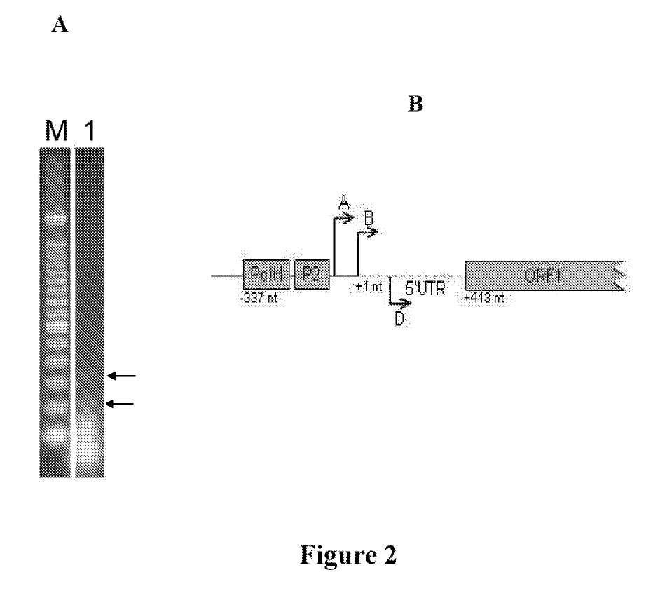

FIG. 2 shows (A) An agarose gel photograph of 5′-RACE using cDNAs from purified recombinant TSV generated in CHO cells. The arrow indicates the cDNA amplicons. (B). A schematic diagram indicating the origin of the transcripts in recombinant TSV as determined by 5′-RACE is shown in panel B. The alphabets indicate the coordinates of each transcript compared to the nucleotide position 1 in the TSV genome. A list of 5′ RACE cDNA clones sequenced and the details of the coordinates of each transcript of recombinant TSV is given in Table 1

The sequence data revealed that there were four classes of transcripts packaged in the mature TSV (Table 1). The predominant class (Group A) contained 56 additional nucleotides at the 5′ end of TSV genome (Table 1). There were three other classes of transcripts that had either 1 (Group B), 5 (Group C) or 78 nucleotide (Group D) deletion at the 5′ end of the viral genome (Table 1). IHHNV P2 promoter is known to be functional in mammalian cells (Dhar et al., unpublished), whereas the polyhedrin promoter is non-functional in mammalian cells. The transcription initiation site (TIS) for the P2 promoter driven gene is 22 nucleotides downstream of the TATA box. Considering this TIS, the TSV mRNA transcribed by the P2 promoter contained an additional 127 nucleotides at the 5′-end of the TSV genome. Therefore, it appears that only 56 out of 127 additional nucleotides were retained during packaging of TSV transcripts in mature virions. There were also TSV transcripts (Groups B, C and D, Table 1) where 127 additional nucleotides along with 1 to 7 nucleotides from TSV 5′ UTR at the 5′ end were deleted before the RNA was packaged into mature virions. Irrespective of the nature of addition or deletions at the 5′-end of rTSV genome, the virus purified from CHO and 293H cells were very virulent and caused high mortality in the injected animals as recoded in the mortality data for Bioassays #1 and #2. A summary of the number of clones sequenced and the corresponding origin of the transcripts is given in Table 1.

TABLE 1
A summary of number of 5′-RACE clones and the origin
of transcripts in CHO and 293H cell derived purified rTSV.

| | 5′-end | | |
|---|---|---|---|
| Origin of RNA used for | coordinates of | # of | Shown |
| 5′ RLM-RACE | the transcripts | clones | on figure |
| Purified virus from mammalian | −56 | 12 | A |
| cells (CHO) infected with | +1 | 5 | B |
| BVTSV + BVORF1/2 | +5 | 5 | B1 |
| | +78 | 3 | D |
| Purified virus from mammalian | −56 | 4 | A |
| cells (293) infected with | +1 | 4 | B |
| BVTSV + BVORF1/2 | +58 | 1 | C |
| | +88 | 3 | D1 |

The nucleotide 1 of TSV genome in the recombinant TSV was considered as +1. The nucleotide position of the transcripts is marked as “+” or “−” depending on whether they originated downstream or upstream of the first nucleotide position of TSV.

### REFERENCES

The contents of all references cited herein are hereby incorporated by reference herein for all purposes.
Bell, T. A. and Lightner, D. V. 1988. A handbook of normal penaeid shrimp histology. World Aquaculture Society, Baton Rouge, La.
Bonami, J. R., Hasson, K. W., Mari, J., Poulos, B. T. and Lightner, D. V. (1997). Taura syndrome of marine penaeid shrimp: characterization of the viral agent. J. Gen. Virol. 78: 313-319.
Christian, P., Carstens, E., Domier, L., Johnson, J., Johnson, K., Nakashima, N., Scotti, P. & van der Wilk, F. (2006). Index of Viruses—Dicistroviridae. In: ICTVdB—The Universal Virus Database, version 4. Biichen-Osmond, C. (Ed), Columbia University, New York, USA. http://www.ncbi.nlm.nih.gov/ICTVdb/Ictv/fs_index.htm
Dhar, A. K., and Allnutt, F. C. T. 2004. Crustacean expression vector, International publication number WO 2005/102041 A2.
Dhar, A. K., Lakshman, D. K., Natarajan, S., Allnutt, F. C. T. and van Beek, N. A. M. 2007. Functional characterization of putative promoter elements from infectious hypodermal and hematopoietic necrosis virus (IHHNV) in shrimp and in insect and fish cell lines. Virus Res. 127: 1-8.
Kuo et al. 1989. Science 244: 362-364.
Lightner, D. V. 1996. A handbook of shrimp pathology and diagnostic procedures for diseases for cultured penaeid shrimp. World Aquaculture Society, Baton Rouge, La.
Lightner, D. V., R. M. Redman, and T. A. Bell. 1983. Infectious Hypodermal and Hematopoietic Necrosis a newly recognized virus disease of penaeid shrimp. J. Invertebr. Pathol. 42: 62 70.
Mari, J., Poulos, B. T., Lightner, D. V. and Bonami, J. R. 2002. Shrimp Taura syndrome virus: economic characterization and similarity with members of the genus Cricket paralysis-like viruses. J. Gen. Virol. 83: 915-926.
Robles-Sikisaka, R., Garcia, D. K., Klimpel, K. R. and Dhar, A. K. (2001). Nucleotide sequence of 3′-end of the genome of Taura syndrome virus of shrimp suggests that it is related to insect picornaviruses. Arch. Viro1.146: 941-952.
Sambrook J, Fritsch E, Maniatis T. (1989) Molecular Cloning: A laboratory Manual, Second Edition, Cold Spring Harbor Press, Cold Spring Harbor.
Shike, H., Dhar, A. K., Burns, J. C., Shimuzu, C., Jousset, F. X., Klimpel, K. R., and Bergoin, M. 2000. Infectious hypodermal and hematopoietic necrosis virus of shrimp is related to mosquito brevidensoviruses. Virology 277: 167-177.
Sittidilokratna, N., Dangtip, S., Cowley, J. A., Walker, P. J. 2008. RNA transcription analysis and completion of the genome sequence of yellow head nidovirus. Virus Res. 136: 157-165.
Tani, H., Limn C-K., Yap, C-C., Onishi, M., Nozaki, M., Nishimune, Y., Okahashi, N., Kitagawa, Y., Watanabe, R., Mochizuki, R., Moriishi, K., and Matsuura, Y. 2003. In vitro and in vivo gene delivery by recombinant baculoviruses. J. Virol. 77: 9799-9808.
van Hulten, M. C. W., Witteveldt, J., Peters S., Kloosterboer, N., Tarchini, R., Fiers, M., Sandbrink, S., Lankhorst, R. K., and Valk, J. M. 2001. The white spot syndrome virus DNA genome sequence. Virology 286: 7-22.
van Regenmortel, M. H. V., Fauquet, C. M., Bishop, D. H. L., Carstens, E. B., Estes, M. K., Lemon, S. M., Maniloff, J., Mayo, M. A., McGeoch, D. J., Pringle, C. R., and Wickner, R. B. (2000). Virus Taxonomy: The Classification and Nomenclature of Viruses. The Seventh Report of the International Committee on Taxonomy of Viruses. Academic Press, San Diego, 1167 pp.
Yang, F., He, J., Lin, X., Li, Q., Pan, D., Zhang, X., and Xu, X. 2001. Complete genome sequence of the shrimp white spot bacilliform virus. J. Virol. 75:11811-11820.
Accession numbers for TSV isolates: AF277675, AY590471, GQ502201, DQ104696 and AY997025.

1. A viral construct comprising a carrier virus, at least one viral promoter of Infectious Hypodermal and Hematopoietic Necrosis Virus (IHHNV) operably linked to at least one inclusion viral genome, gene or fragment thereof, wherein the inclusion viral genome is different from that of the carrier virus.

2. The viral construct of claim 1, wherein the viral promoter of IHHNV is selected from the group consisting of P2 and P61.

3. The viral construct of claim 1, wherein the carrier virus is a baculovirus.

4. The viral construct of claim 1, wherein the inclusion viral genome is Hepatitis C Virus (HCV).

5. A method to produce a functional virus in a heterologous system, the method comprising:
preparing a nucleotide sequence of a virus;
linking a IHHNV promoter sequence upstream of the 5′ end of the nucleotide sequence to drive the transcription of the nucleotide sequence;
inserting the linked sequences into a transfecting carrier virus; and
infecting and culturing a host cell for expression therein of the linked sequences and carrier virus.

6. The method of claim 5, wherein the virus is an RNA or DNA virus.

7. The method of claim 5, wherein the IHHNV promoter is selected from the group consisting of P2 and P61.

8. The method of claim 5, wherein the carrier virus is a baculovirus.

9. The method of claim 5, wherein the inclusion viral genome is HCV.

10. The method of claim 5, wherein the host cells do not support the replication of baculovirus.

11. A host cell comprising the viral construct of claim 1.

12. A method of expressing recombinant proteins in a heterologous culture system, the method comprising:
preparing a nucleotide sequence encoding the proteins,
linking a IHHNV promoter sequence upstream of the 5′ end of the nucleotide sequence encoding the proteins to drive the transcription of the nucleotide sequence and forming a linked sequence;
inserting the linked sequence into a transfecting carrier virus; and
infecting and culturing a mammalian host cell for expression therein of the proteins and wherein the carrier virus is not expressed.

13. The method of claim 12, wherein the proteins are from a virus.

14. The method of claim 12, wherein the IHHNV promoter is selected from the group consisting of P2, P61 and MID.

15. The method of claim 12, wherein the carrier virus is a baculovirus.

16. The method of claim 12, wherein the encoded proteins are from the virus TSV.
